(19) United States
(12) Patent Application Publication
(10) Pub. No.: US 2014/0248310 A1
(43) Pub. Date: Sep. 4, 2014

(54) CATIONIC METAL OXIDES FOR USE AS VACCINE ADJUVANTS

(71) Applicant: Frank A. Robey, Bethesda, MD (US)

(72) Inventor: Frank A. Robey, Bethesda, MD (US)

(21) Appl. No.: 14/134,378

(22) Filed: Dec. 19, 2013

Related U.S. Application Data
(60) Provisional application No. 61/848,122, filed on Dec. 20, 2012.

(51) Int. Cl. A61K 39/39; A61K 39/02; A61K 39/145

(52) U.S. Cl. CPC A61K 39/39; A61K 39/145; A61K 39/105; A61K 2039/55555
USPC 424/204.1; 556/173; 556/10; 556/405; 556/425; 556/413; 424/278.1; 534/15; 424/277.1; 424/234.1; 424/274.1

(57) ABSTRACT

Adjuvant and immunological vaccine compositions comprising modified, cationic metal oxides are disclosed, including methods of making modified, cationic metal oxides and methods of using the modified metal oxides in vaccine formulations and regimens.

### CROSS-REFERENCE TO RELATED APPLICATIONS

This application claims benefit of priority to U.S. provisional application No. 61/848,122 filed on Dec. 20, 2012, which is incorporated by reference in its entirety.

### FIELD OF INVENTION

This invention relates to immunological adjuvant compositions used for antibody preparations and as a component of vaccines.

### BACKGROUND

Immunological adjuvants are agents capable of stimulating the immune system to elicit an increased immunological response to antigenic substrates. Though immunological adjuvants are co-administered with antigenic substrates, the adjuvants are not antigenic themselves.

The adjuvants potentiate non-specifically the ensuing immune response. A principal purpose for employment of an immunological adjuvant is to achieve more durable humoral or cell-mediated immunity of a high level by employing lower levels of an antigen with fewer numbers of doses than could be achieved by administering the equivalent aqueous antigen. Adjuvants are used in combination with non-living agents (in place of living microorganisms), for the preparation of vaccines. Adjuvants may also increase the effective immune response against low or nonimmunogenic tumor cells or cells infected with intracellular agents that are already present in the body and are not adequately checked by naturally elicited immune responses.

Adjuvant agents include large macromolecular entities (“LME class”) and small molecular entities (“SME class”). Members of the LME class include proteins and polysaccharides, such as albumin and bacterial cell wall components. Members of the SME class include aluminum salts and squalene.

Aluminum-containing adjuvants today remain the mainstay of vaccine formulations used in both humans and animals. However, little is known about the biological mechanisms the aluminum-containing adjuvants use to enhance the immune response.

The physical chemical properties of aluminum-containing adjuvants have been studied extensively. The materials used are aluminum hydroxide, aluminum phosphate and aluminum sulfate, all of which are anionic at physiological pH's. In addition, it is well-known that aluminum-adjuvanted vaccines should not be exposed to freezing conditions, conditions which have been shown to denature the formulations rendering them inactive. (WHO, Safe Vaccine Handling, Cold Chain and Immunizationss, World Health Organization, Geneva, 1996; Galazka, A. et al., Thermostability of Vaccines, World Health Organization, Geneva, 1996.

Frey et al. (Bioconjugate Chem. 8:424-433 (1997)) used modified aluminum oxide (“MAO”) as an adjuvant-carrier for small oligopeptides. One such MAO composition, 3-aminopropyltriethoxysilyl α-Al₂O₃ nanoparticle, has been used as a starting reagent for further derivatization and use as a carrier for oligopeptide antigens derived from the HIV-1 viral envelope glycoprotein gp120 molecule. In such applications, the MAO composition is covalently coupled to the antigen, because it was widely thought that the MAO composition only worked as a covalent carrier adjuvant.

Cationic aluminum oxide (MAO) as an adjuvant has not been reported. MAO, described here along with other cationic metallic oxides used as adjuvants, was found to have the following unexpected and surprising properties: (1) the ability to bind and precipitate immunogens that contain anionic amino acids, such as glutamic acid and aspartic acid, at physiological pH; (2) stability as dry powders; and (3) stability at a range of temperatures. In addition, vaccines formulated with MAO can be administered either mucosally (intranasally or orally or intravaginally, or intrarectally) or subcutaneously

### SUMMARY

In one respect, the invention relates to adjuvant agent comprising a modified metal oxide, on the proviso the modified metal oxide is not a 3-aminopropyltriethoxysilyl α-Al₂O₃ nanoparticle.

In a second respect, the invention relates to immunological composition that includes an adjuvant agent and an immunogen. The adjuvant agent comprises a modified metal oxide, wherein the adjuvant agent is not covalently attached to the immunogen.

In a third respect, the invention relates to a method of eliciting an immunogenic response in a host to an immunogen. The method includes the step of administering an effective amount of the immunogen. The immunogen is admixed with an adjuvant agent to form a non-covalent complex between the immunogen and the adjuvant agent, wherein the adjuvant agent comprises a modified metal oxide.

In a fourth respect, the invention relates to a vaccine directed against an antigenic substrate that includes an adjuvant agent and the antigenic substrate. The adjuvant agent includes a modified metal oxide comprising a 3-aminopropyltriethoxysilyl α-Al₂O₃ nanoparticle or a 3-aminopropyltriethoxysilyl α-Al₂O₃ microparticle. The adjuvant agent is not covalently attached to the antigenic substrate.

In a fifth respect, the invention relates to a method of vaccinating a subject against an antigenic substrate. The method includes the step of administering a vaccine directed against the antigenic substrate to the subject. The vaccine includes an adjuvant agent and the antigenic substrate. The adjuvant agent includes a modified metal oxide, wherein the adjuvant agent is not covalently attached to the antigenic substrate.

In a sixth respect, the invention relates to an adjuvant agent includes a compound having formula (I): X—[Y—(Z)ₘ]ₙ (I), wherein: X is a particle comprising a metal oxide; Y is a linker; and Z is a chemical moiety having a positive charge at physiological pH; further wherein: m is an integer from 1 to 5; and n is an integer or a fraction thereof from 0.5 to 50, on the proviso X—[Y—(Z)ₘ]ₙ is not a 3-aminopropyltriethoxysilyl α-Al₂O₃ nanoparticle.

In a seventh respect, the invention relates to an immunological composition that includes an adjuvant agent and an immunogen. The adjuvant agent includes a compound having formula (I): X—[Y—(Z)ₘ]ₙ (I), wherein: X is a particle comprising a metal oxide; Y is a linker; and Z is a chemical moiety having a positive charge at physiological pH; further wherein: m is an integer from 1 to 5; and n is an integer or a fraction thereof from 0.5 to 50. The adjuvant agent is not covalently attached to the immunogen.

In an eighth respect, the invention relates to a method of eliciting an immunogenic response in a host to an immunogen that includes the step of administering an effective amount of the immunogen, wherein said immunogen is admixed with an adjuvant agent to form a non-covalent complex between the immunogen and the adjuvant agent. The adjuvant agent comprises a compound having formula (I), X—[Y—(Z)ₘ]ₙ (I), wherein: X is a particle comprising a metal oxide; Y is a linker; and Z is a chemical moiety having a positive charge at physiological pH; further wherein m is an integer from 1 to 5; and n is an integer or a fraction thereof from 0.5 to 50.

In a ninth respect, the invention relates to a vaccine directed against an antigenic substrate. The vaccine includes an adjuvant agent and the antigenic substrate. The adjuvant agent comprises a compound having formula (I), X—[Y—(Z)ₘ]ₙ (I), wherein: X is a particle comprising a metal oxide; Y is a linker; and Z is a chemical moiety having a positive charge at physiological pH; further wherein m is an integer from 1 to 5; and n is an integer or a fraction thereof from 0.5 to 50. The adjuvant agent is not covalently attached to the antigenic substrate.

In a tenth respect, the invention relates to a method of vaccinating a subject against an antigenic substrate that includes the step of administering a vaccine directed against the antigenic substrate to the subject. The vaccine includes an adjuvant agent and the antigenic substrate. The adjuvant agent comprises a compound having formula (I), X—[Y—(Z)ₘ]ₙ (I), wherein: X is a particle comprising a metal oxide; Y is a linker; and Z is a chemical moiety having a positive charge at physiological pH; further wherein m is an integer from 1 to 5; and n is an integer or a fraction thereof from 0.5 to 50. The adjuvant agent is not covalently attached to the antigenic substrate.

### BRIEF DESCRIPTION OF THE DRAWINGS

FIG. 1 depicts a synthetic reaction scheme for producing modified metal oxides, where 101 represents an exemplary metal oxide particle (Al₂O₃); 102 represents an exemplary modification reagent (3-aminopropyltriethoxysilylane); 103 represents an exemplary linker (1-propyltriethoxysilyl); 104 represents an exemplary chemical moiety having a positive charge at physiological pH (a primary amine); and 105 represents the resultant modified metal oxide, as exemplified by 3-aminopropyltriethoxysilyl-Al₂O₃.

FIG. 2a illustrates anti-LAH peptide antigen serum IgG antibody levels from mice inoculated via intranasal administration (“i/n”) with immunological compositions of LAH peptide antigen covalently coupled to aluminum oxide (“LAH complex i/n”); with immunological compositions of LAH peptide antigen bound electrostatically to cationic aluminum oxide, MAO (“LAH plus i/n”); with immunological compositions of LAH peptide antigen in the absence of any aluminum oxide (“LAH free i/n”) or with Hepatitis C viral peptide antigen covalently coupled to aluminum oxide (“HCV peptide complex i/n”). Mouse IgG levels were determined on 1:100 dilutions of serum samples using an enzyme linked immunoadsorbant assay ELISA.

FIG. 2b illustrates anti-LAH peptide antigen IgG antibody levels from serum of mice inoculated via subcutaneous administration (“s/c”) with immunological compositions of LAH peptide antigen covalently coupled to a modified metal oxide adjuvant (“LAH complex s/c”); with immunological compositions of LAH peptide antigen bound electrostatically to free MAO (“LAH plus s/c”); with immunological compositions of LAH peptide antigen alone (“LAH free s/c”); or with Hepatitis C viral peptide antigen covalently coupled to a modified metal oxide adjuvant (“HCV peptide complex s/c”).

FIG. 2c illustrates titers of anti-LAH peptide antigen IgG antibody levels from serum of mice inoculated via either intranasal administration (“i/n”) or subcutaneous administration (“s/c”) with immunological compositions of LAH peptide antigen covalently coupled to a modified metal oxide adjuvant (“LAH complex i/n” and “LAH complex s/c”); with immunological compositions of LAH peptide antigen bound electrostatically to modified metal oxide adjuvant (“LAH plus i/n” and “LAH plus s/c”), with immunological compositions of LAH peptide antigen alone (“LAH free i/n” and “LAH free s/c”) in animals after receiving the primary immunization and two secondary booster immunizations with these respective immunological compositions. Results indicate that the IgG produced against LAH was the same regardless of whether the LAH was covalently coupled to MAO or ionically coupled to MAO. The greatest number of mice with the highest titers were those that were immunized with LAH peptide electrostatically bound to MAO.

FIG. 3 illustrates levels of bacterium-specific 16S rDNA per gram animal host tissue following infection of Helicobacter pylori (“H. pylori”) animals after the animals received intranasal administration of 3-aminopropyltriethoxysilyl Al₂O₃ adjuvant alone (channel A: “MAO”), a first surface antigen peptide from H. pylori (“Hp2”) covalently complexed to Al₂O₃ (“AO”) (channel B: “AO−Hp2”), a second surface antigen peptide from H. pylori (“Hp10”) covalently bound to Al₂O₃ (“AO”) (channel C: “AO−Hp10”), a mixture of the two aforementioned surface antigen peptides from H. pylori (“Hp2/10”) covalently complexed to Al₂O₃ (“AO”) (channel D: “AO−Hp2/10”), the first surface antigen peptide from H. pylori in an admixture with free, uncoupled modified metal oxide adjuvant (channel E: “MAO+Hp2”), the second surface antigen peptide from H. pylori in an admixture with free, uncoupled modified metal oxide adjuvant (channel F: “MAO+Hp10”); or after the animals received subcutaneous administration of MAO (channel G), AO−Hp2/10, or nothing (channel H: “Naïve”). Infection of vaccinated mice with Helicobacter pylori (HP) as measured by PCR determination of HP DNA from stomachs of HP inoculated mice. Groups of 5 mice were vaccinated with various constructs shown along the X-axis. For example, for A, 5 mice were immunized intranasally with MAO and for B, 5 mice were immunized intranasally with Al₂O₃ to which a peptide from HP, designated here as HP-2, was covalently conjugated.

### DETAILED DESCRIPTION OF THE INVENTION

Applicant has discovered a surprising property of modified metal oxides in their use as immunological adjuvants for vaccines. Though it was recognized that modified metal oxides, such as MAO, could be used as adjuvant-carriers for small molecule antigens, it was widely believed that covalent coupling of MAO to the antigen was required for effective immunological response to the antigen. Applicant discovered that covalent coupling of the MAO to the antigen is not required for generating a robust immunological response to the antigen. This unexpected and surprising discovery means that MAO and other modified metal oxides can be used as adjuvants simply by mixing them with antigen prior to administration. A further advantage to the use of MAO and other modified metal oxides as uncoupled, “free,” adjuvants is that further modification or derivatization of the modified metal oxides to accommodate specific coupling reactions to antigens is avoided altogether. This provides the additional advantage that modified metal oxide adjuvant/antigen vaccine compositions can be prepared in more pure form and free of potentially dangerous contaminants associated with coupling chemistry that would be required to prepare covalently-coupled modified metal oxide-antigen vaccine compositions. Further economical advantages exist to use of modified metal oxides as uncoupled, free adjuvants owing to the fact that the costs of using modification and derivatization chemistries and the attendant costs of subsequent purification of the resultant products are eliminated. Finally, the resultant modified metal oxides are remarkably robust and stable, thereby rendering them ideal immunological adjuvants amenable for vaccine administration in the field and in third world countries.

### Modified Metal Oxide Particles

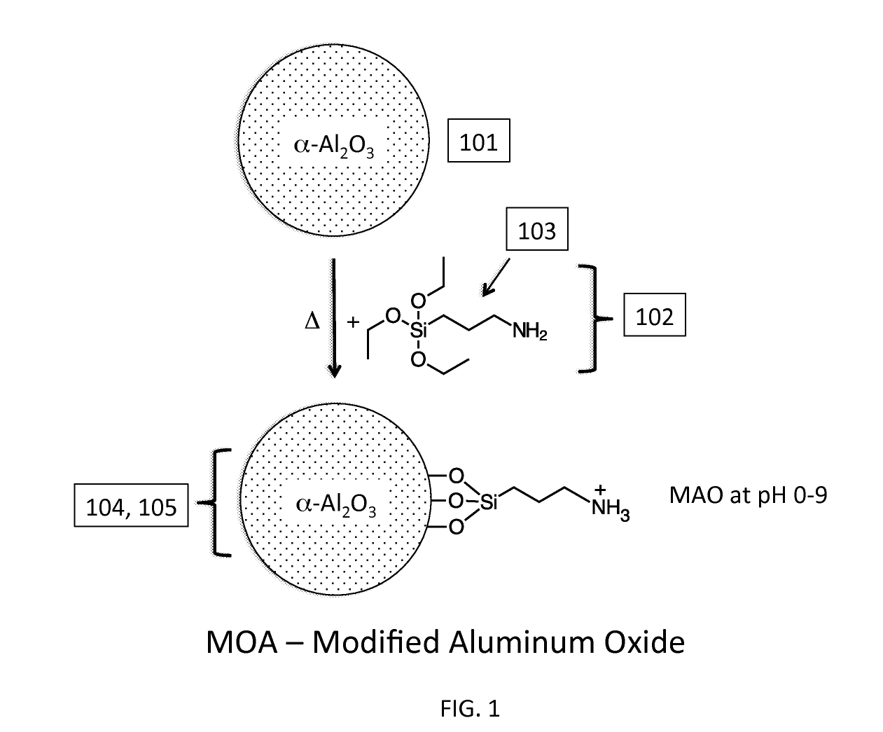

FIG. 1 illustrates a synthesis of an exemplary modified metal oxide, 3-aminopropyltriethoxysilyl α-Al₂O₃ nanoparticle. The synthesis uses a metal oxide 101 and a modification reagent 102. The metal oxide 101 includes one or more metals selected from aluminum, titanium, zirconium, calcium, magnesium, silicon, yttrium, scandium, lanthanum, among others.

Metal oxide 101 having a particulate form is preferred. Metal oxide 101 can have a size (expressed as average nominal mean diameter) in the nanoparticle or microparticle ranges. Preferred sizes of nanoparticles for metal oxide 101 include sizes from about 5 nm to about 1000 nm. More preferred sizes of nanoparticles for metal oxide 101 include sizes from about 20 nm to about 300 nm. Highly preferred sizes of nanoparticles for metal oxide 101 include sizes from 20 nm to about 50 nm, including about 20 nm, about 40 nm, and about 50 nm, among other sizes. Preferred sizes of microparticles for metal oxide 101 include sizes from about 1 μm to about 100 μm. More preferred sizes of microparticles for metal oxide 101 include sizes from about 2 μm to about 50 μm. Highly preferred sizes of microparticles for metal oxide 101 include sizes from about 3 μm to about 30 μm, including about 3 μm, about 5 μm, and about 30 μm, among other sizes. Sizes of nanoparticles and microparticles of metal oxide 101 within the broadest preferred ranges are available from commercial vendors or can be prepared by methods known in the art (for example, milling, grinding or other particulate size reducing methods).

Metal oxide 101 can be of any regular or irregular shape as starting material for the synthesis of modified metal oxides of FIG. 1. Metal oxide 101 having a substantially spherical particle shape is generally preferred.

The modification reagent 102 includes a linker 103 and a chemical moiety. The linker 103 includes a reactive moiety capable of forming at least one bond to the metal oxide 101. A preferred modification reagent 102 includes a linker 103 that includes a reactive moiety having a metal ether composition. Examples of such metal ether compositions include triethoxysilane, triethoxysilane, and tripropoxysilane, among others. A highly preferred modification reagent 102 includes a linker 103 that is linked to a reactive moiety, triethoxysilane. The function of the reactive moiety is to couple linker 103 to metal oxide 101.

Preferred modification reagent 102 includes a linker 103 that includes a reactive moiety having a metal ether composition and further includes additional chemical spacer motifs, such as an alkyl or alkenyl group. Preferred chemical spacer motifs of linker 103 include straight or branched chain alkyl groups having 1-20 carbons and optionally contain additional chemical modifications or substituents to modulate the ADME characteristics of the resultant modified metal oxides. A highly preferred linker 103 is 1-propyltriethoxysilane.

The chemical moiety 104 has a positive charge at physiological pH. Preferred examples of chemical moiety 104 include a primary amine, a secondary amine and a tertiary amine. A highly preferred chemical moiety 104 is an alkyl primary amine.

In certain preferred embodiments of modification reagent 102, a plurality of chemical moiety 104 can exist in modification reagent 102 that permits a plurality of positive charges at physiological pH. In highly preferred embodiments of modification reagent 102, a preferred plurality of chemical moiety 104 can range from two to ten, with a highly preferred plurality of chemical moiety 104 being two to five.

As stated above, preferred modification reagent 102 includes a linker 103 and a chemical moiety 104. Highly preferred examples of modification reagent 102 include an amino alkyltriethoxysilane, such as 3-aminopropyltriethoxysilane.

Referring again to FIG. 1, metal oxide 101 is activated by heating at an appropriate elevated temperature (for example, above 85° C.) and thoroughly washed with water prior to drying. Dried, surface-activated metal oxide 101 is then combined with an appropriate modification reagent 102 at an appropriate elevated temperature (for example, above 130° C.) to promote reaction between metal oxide 101 and modification reagent 102 and to form modified metal oxide 105. The resultant product is washed with water, dried, and stored under dessicated conditions.

In certain modified metal oxide 105 compositions prepared according to the synthesis scheme presented in FIG. 1, complex mixtures of modified metal oxides can form. In preferred compositions, modified metal oxides include only one modification where the stoichiometry of metal oxide 101: modification reagent 102 is 1:1 in the resultant modified metal oxide 105. In other preferred modified metal oxide 105 compositions, the stoichiometry of metal oxide 101: modification reagent 102 can vary from 1:0.5 to 1:50 and mixtures thereof.

Likewise, the resultant modified metal oxide 105 can have a size greater than metal oxide 101 owing to the addition of one or more modification reagent 102 per molecule of metal oxide 101 in modified metal oxide 105. Accordingly, should specific size classes of resultant modified metal oxide 105 be desired (for example, the nominal size of unmodified metal oxide 101), particle size reduction procedures available to one skilled in the art can be employed (such as milling, grinding, among others) to reduce the resultant modified metal oxide 105 (or mixtures thereof) to such a size.

In other preferred embodiments, modified metal oxide 105 includes compositions having formula (I):

X—[Y—(Z)ₘ]ₙ (I),

wherein

X comprises metal oxide 101, Y comprises linker 103 and Z comprises chemical moiety 104. The components Y and Z together constitute the post-reaction product of modification reagent 102. The terms m and n represent the number of Z and Y—(Z)ₘ, in formula (I), respectively. The term m and n preferably represent whole integers, where m ranges from 1-10 and n ranges from 1-50. In other embodiments, n can represent fractional values less than about 1.00 that fall within the range from about 0.10 to about 0.95, including about 0.10, about 0.25, about 0.33, about 0.40, about 0.50, about 0.75 and about 0.95, as well as other fractional values with that range. In other embodiments, n can represent fractional values greater than a whole integer. For example, fractional values falling between 1.00 and 2.00 can include about 1.25, about 1.33, about 1.50, about 1.67, about 1.75 and about 1.90, as well as other fractional values with that range. Fractional values for n arise in mixtures owing to one or more metal oxides 101 forming complexes with a common modification reagent 102, as well as one or more modification reagents 102 forming complexes with a common metal oxide 101.

Under certain conditions of reaction according to FIG. 1, preferred embodiments of modified metal oxide 105 include compositions having formula (I), wherein different species having different values of m and n may exist within the same mixture. For example, mixtures of modified metal oxide 105 can include two or more different compositions having formula (I), such as X—[Y—(Z)₁]₁, X—[Y—(Z)₁]₂, and X—[Y—(Z)₁]₃, among others.

Modified metal oxide 105 can be prepared by alternative routes other than that depicted in FIG. 1, as would be understood to one skilled in the art. For example, a alternative modification reagent can include a linker 103 and a reactive moiety other than chemical moiety 104. Following coupling of metal oxide 101 to the alternative modification reagent to form modified metal oxide precursor, the reactive moiety can be converted to chemical moiety 104 by modification of reactive moiety with another chemical reagent to form modified metal oxide 105 having chemical moiety 104. The preferred synthetic route for preparing modified metal oxide 105, however, is one that requires the fewest reagents and the fewest chemical steps, such as that illustrated in FIG. 1.

Accordingly, metallic oxides suitable for use in modified metal oxide 105 include at least one selected from the group consisting of aluminum oxide (Al₂O₃), titanium dioxide (TiO₂), zirconium dioxide (ZrO₂), hydroxyapatite (Ca₅(PO₄)₃(OH)), silicon dioxide (SiO₂), magnesium oxide (MgO), yttrium oxide (Y₂O₃), scandium oxide (Sc₂O₃), lanthanum oxide (La₂O₃) and mixed oxides of the above, as the preferred embodiments. Other metallic oxides may also be utilized in modified metal oxide 105 provided that these are biocompatible and provide inorganic particles suitable for use in an admixture with an antigen, hapten or immunogen. Of these, aluminum oxide particles are the most desirable choice for in vivo uses and applications, especially when the admixture composition is to be employed either as an immunogen or a vaccine.

As explained above, it is often desirable to reduce the particle size of the resultant modified metal oxide preparations prior to their use as immunological adjuvants. Particle size reduction can be achieved in a number of ways understood to one skilled in the art. Examples include grinding, milling (for example, jet milling, wet ball milling), micronization, nanonization, microfluidization, and high-pressure homogenization technologies. Commercial instruments and processes directed to these various techniques are available for achieving particle size reduction and particle size uniformity. The resultant sizes and dimensional attributes of the resultant particle population can be determined using methods well known in the art.

Preferred modified metal oxide particle sizes can be selected based upon the modality of administration to subject. For injectable pharmaceutical and immunological compositions, the modified metal oxide particles are preferably less than about 100 nm in diameter. For orally- and intranasally-administered, the modified metal oxide particles have a preferable size ranging from about 100 nm to about 1 μm in diameter.

Immunological and Vaccine Compositions with Modified Metal Oxides.

Admixtures of immunogens or antigen substrates from a variety of sources (e.g., cancer antigens, viral antigens, bacterial antigens, or fungal antigens) in the form of different macromolecular species (for example, peptides, proteins, nucleic acids and carbohydrates) and modified metal oxides having a positive charge at physiological pH (for example, 3-aminopropyltriethoxysilyl α-Al₂O₃ particles, among others) are prepared by mixing these materials in aqueous media and at pH ranging from 6-8 where the resultant modified metal oxides would be predominately protonated and thus positively charged. Negatively-charged materials will bind to resultant modified metal oxides and such binding can be disrupted by either raising the pH to above pH 10 to deprotonate chemical moiety having a positive charge at physiological pH on the modified metal oxide or lowering the pH to below pH 3 to protonate the carboxyl on the modified metal oxide binder. Exemplary applications of such admixtures at physiological pH and their use in method for antibody production and vaccination regimens are disclosed below.

Cancer antigens amenable for use as immunogens and antigenic substrates with the modified metal oxide adjuvants include breast cancer, lung cancer, prostate cancer, bladder cancer, liver cancer, kidney cancer, stomach cancer, pancreatic cancer, cervical cancer, vaginal cancer, bone cancer, other forms of sarcoma, lymphoma and leukemia, among others. Immunological compositions include the modified metal oxide particle as adjuvant agent and the cancer immunogen, wherein the modified metal oxide particle is not covalently attached to the cancer immunogen. Vaccines directed against a cancer can be prepared that include a modified metal oxide particle as adjuvant agent and a cancer-specific antigenic substrate, wherein the modified metal oxide particle is not covalently attached to the cancer-specific antigenic substrate. Methods of eliciting an immunogenic response in a host to a cancer immunogen are also contemplated wherein one administers an effective amount of the cancer immunogen to the patient. Prior to administration, the cancer immunogen is admixed with an adjuvant agent to form a non-covalent complex between the immunogen and the adjuvant agent, and wherein the adjuvant agent comprises a modified metal oxide. Likewise, methods vaccinating a subject against a cancer antigenic substrate, are contemplated wherein the methods include the step of administering a vaccine directed against the cancer antigenic substrate to the subject. The vaccine includes an adjuvant agent and the cancer antigenic substrate, wherein the adjuvant agent includes a modified metal oxide, and wherein the adjuvant agent is not covalently attached to the cancer antigenic substrate. Viral antigens amenable for use as immunogens and antigenic substrates with the modified metal oxide adjuvants include influenza A, B, and C viruses and other Orthomyxoviridae, hepatitis A and B viruses and other Hepadnaviridae, HTLV-II and HTLV-II leukemia viruses, HIV-1 AIDS virus, paramyxovirus, adenovirus, HSV-1 and HSV-2 herpes simplex viruses, Rhinovirus, Norwalk Virus, Respiratory Syncytial Virus, Picornavirus, Coxsackievirus, Polioviru, Varicella zoster virus, Epstein-Barr virus, Cytomegalovirus, Kaposi's sarcoma-associated herpesvirus, Flavivirus, Rhabdoviridae, Flaviviridae, Dengue fever, West Nile virus, Yellow fever, Measles virus, Mumps virus, Human parainfluenza viruses, Metapneumovirus, Papillomaviridae, Rhabdoviridae, Rabies virus, Togaviridae, Rubella virus, Parvoviridae, Human bocavirus, and Parvovirus B19, among others. Immunological compositions include the modified metal oxide particle as adjuvant agent and the viral immunogen, wherein the modified metal oxide particle is not covalently attached to the viral immunogen. Vaccines directed against a virus or viral infection can be prepared that include a modified metal oxide particle as adjuvant agent and a virus-specific neutralizing antigenic substrate, wherein the modified metal oxide particle is not covalently attached to the virus-specific antigenic substrate. Methods of eliciting an immunogenic response in a host to a viral immunogen are also contemplated wherein one administers an effective amount of the viral immunogen to the patient. Prior to administration, the viral immunogen is admixed with an adjuvant agent to form a non-covalent complex between the viral immunogen and the adjuvant agent, and wherein the adjuvant agent comprises a modified metal oxide. Likewise, methods vaccinating a subject against a viral antigenic substrate are contemplated wherein the methods include the step of administering a vaccine directed against the viral antigenic substrate to the subject. The vaccine includes an adjuvant agent and the viral antigenic substrate, wherein the adjuvant agent includes a modified metal oxide, and wherein the adjuvant agent is not covalently attached to the viral antigenic substrate.

Bacterial antigens amenable for use as immunogens and antigenic substrates with the modified metal oxide adjuvants include Bacillus anthracis, Helicobacter pylori, Rickettsia, Chlamydia trachomatis, Neisseria gonorrhoeae, Treponema pallidum, Ureaplasma urealyticum, Haemophilus ducreyi, Escherichia coli, Staphylococcus saprophyticus, Haemophilus influenzae, Streptococcus pneumoniae, Neisseria meningitidis, Streptococcus agalactiae, Listeria monocytogenes, Mycobacterium pneumoniae, Chlamydia [Chlamydophila] pneumoniae, Legionella pneumophila, Mycobacterium tuberculosis, Pseudomonas aeruginosa, Staphylococcus aureus, Streptococcus pyogenes, Campylobacter jejuni, Salmonella, Shigella, Clostridium difficile, Burkholderia cenocepacia, and Mycobacterium avium, among others. Immunological compositions include the modified metal oxide particle as adjuvant agent and the bacterial immunogen, wherein the modified metal oxide particle is not covalently attached to the bacterial immunogen. Vaccines directed against a bacterium or bacterial infection can be prepared that include a modified metal oxide particle as adjuvant agent and a bacteria-specific antigenic substrate, wherein the modified metal oxide particle is not covalently attached to the bacterium-specific antigenic substrate. Methods of eliciting an immunogenic response in a host to a bacterial immunogen are also contemplated wherein one administers an effective amount of the bacterial immunogen to the patient. Prior to administration, the bacterial immunogen is admixed with an adjuvant agent to form a non-covalent complex between the bacterial immunogen and the adjuvant agent, and wherein the adjuvant agent comprises a modified metal oxide. Likewise, methods vaccinating a subject against a bacterial antigenic substrate are contemplated wherein the methods include the step of administering a vaccine directed against the bacterial antigenic substrate to the subject. The vaccine includes an adjuvant agent and the bacterial antigenic substrate, wherein the adjuvant agent includes a modified metal oxide, and wherein the adjuvant agent is not covalently attached to the bacterial antigenic substrate.

Fungal antigens amenable for use as immunogens and antigenic substrates with the modified metal oxide adjuvants include Candida albicans, Aspergillus fumigatus, Aspergillus flavus, Aspergillus clavatus, Cryptococcus neoformans, Cryptococcus laurentii, Cryptococcus albidus, Cryptococcus gattii, Histoplasma capsulatum, Pneumocystis jirovecii, Pneumocystis carinii, and Stachybotrys chartarum, among others. Immunological compositions include the modified metal oxide particle as adjuvant agent and the fungal immunogen, wherein the modified metal oxide particle is not covalently attached to the fungal immunogen. Vaccines directed against a fungal infection can be prepared that include a modified metal oxide particle as adjuvant agent and a fungus-specific antigenic substrate, wherein the modified metal oxide particle is not covalently attached to the fungus-specific antigenic substrate. Methods of eliciting an immunogenic response in a host to a fungal immunogen are also contemplated wherein one administers an effective amount of the bacterial immunogen to the patient. Prior to administration, the fungal immunogen is admixed with an adjuvant agent to form a non-covalent complex between the fungal immunogen and the adjuvant agent, and wherein the adjuvant agent comprises a modified metal oxide. Likewise, methods vaccinating a subject against a fungal antigenic substrate are contemplated wherein the methods include the step of administering a vaccine directed against the fungal antigenic substrate to the subject. The vaccine includes an adjuvant agent and the fungal antigenic substrate, wherein the adjuvant agent includes a modified metal oxide, and wherein the adjuvant agent is not covalently attached to the fungal antigenic substrate.

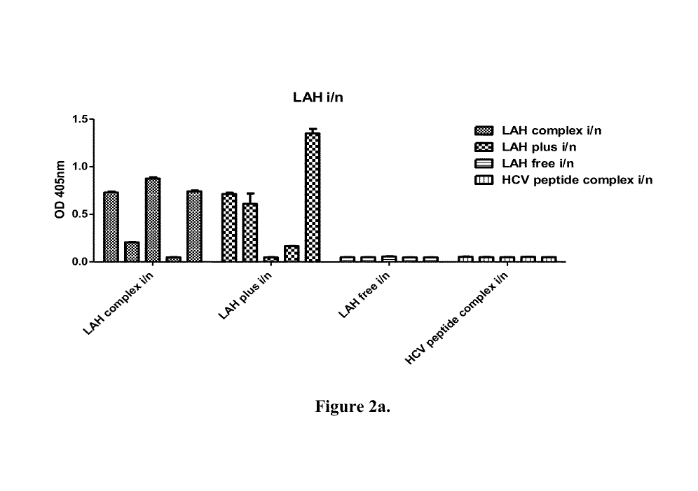

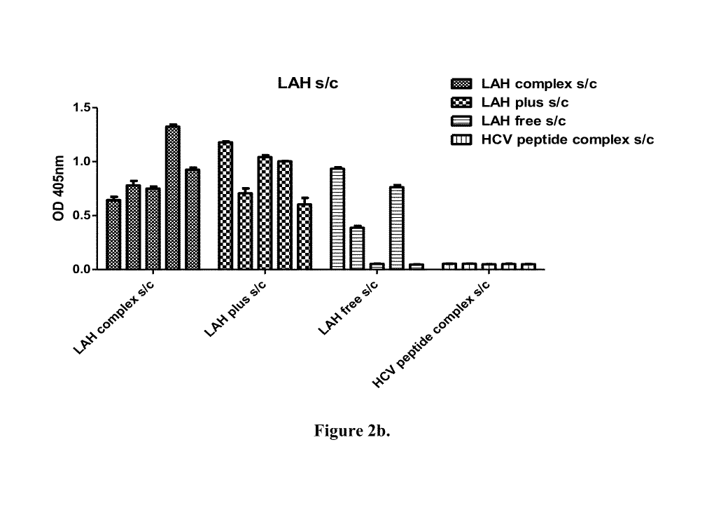

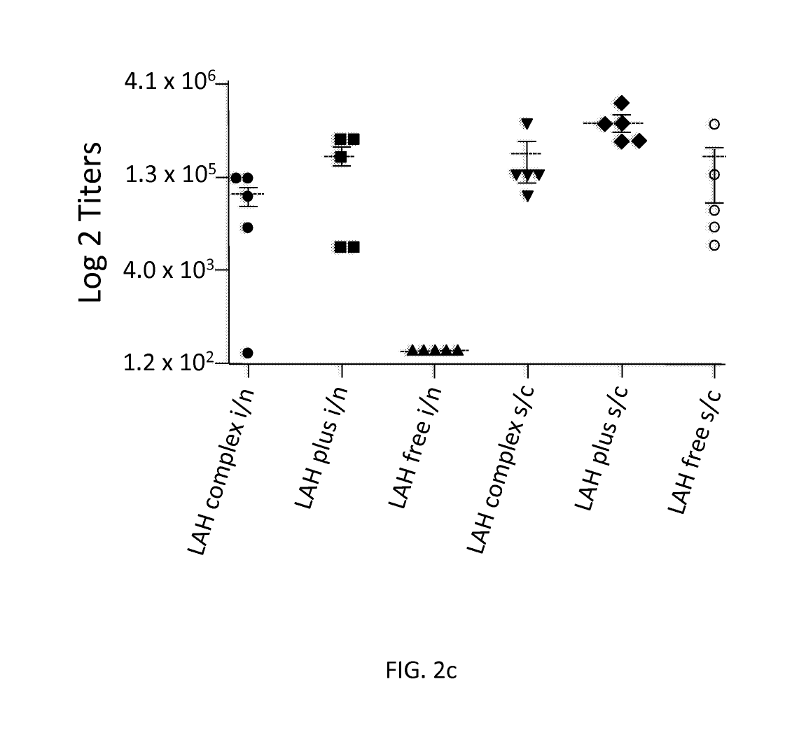

The efficacy of immunological compositions that include the LAH peptide from influenza virus hemagglutinin protein and modified metal oxide adjuvants is demonstrated by the ability to elicit a serum IgG antibody response against the LAH peptide viral antigen. Referring to FIG. 2a, Anti-LAH IgG antibody levels in serum obtained from mice inoculated intranasally with an immunological composition that included LAH peptide antigen and free modified metal oxide adjuvant (3-aminopropyltriethoxysilyl α-Al₂O₃ particles) was quite robust, and as effective, if not superior, to the corresponding immune response in animals that received LAH peptide antigen-modified metal oxide covalent conjugates as an immunological composition via the same administrative route (see FIG. 2a, compare “LAH complex i/n” [includes LAH peptide antigen-modified metal oxide covalent complex] vs. “LAH plus i/n” [includes LAH peptide antigen and uncoupled, free modified metal oxide]). FIG. 2a also demonstrates the overall immunological effectiveness of intranasal administration of immunological compositions including the LAH peptide antigen in the presence of a modified metal oxide adjuvant over corresponding immunological compositions containing LAH peptide antigen in the absence of the modified metal oxide adjuvant (see FIG. 2a, compare “LAH complex i/n” [includes LAH peptide antigen-modified metal oxide covalent complex] and “LAH plus i/n” [includes LAH peptide antigen and uncoupled, free modified metal oxide] vs. “LAH free i/n” [LAH peptide antigen without modified metal oxide adjuvant]). FIG. 2b illustrates similar anti-LAV IgG antibody profiles for animals subcutaneous administrations of immunological compositions of LAH peptide antigen with the modified metal oxide adjuvants as illustrated in FIG. 2a. Finally, FIG. 2c shows that the robust immunological response can be sustained in animals receiving immunological compositions (a primary inoculation and two subsequent boosts) containing the uncoupled, free modified metal oxide adjuvant as compared to LAH peptide antigen without the modified metal oxide adjuvant. In this regard, the immunological compositions containing uncoupled, free modified metal oxide adjuvant were as effective (if not more effective) than immunological compositions containing the modified metal oxide adjuvant in covalent complex with the LAH peptide antigen).

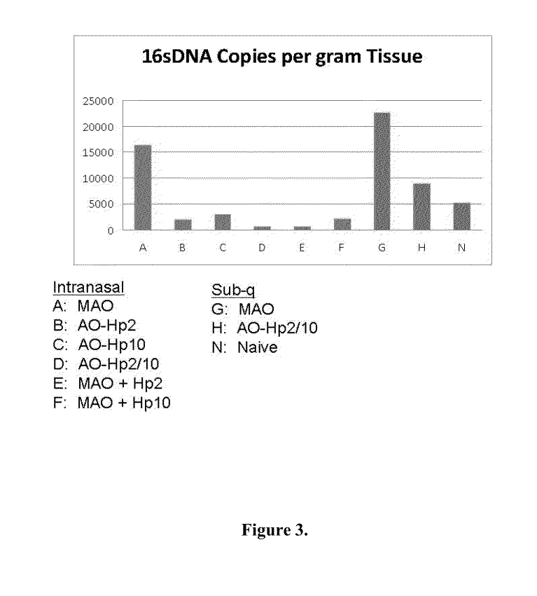

The efficacy of immunological compositions that include two peptide surface antigens (denoted as “Hp2” and “Hp 10” in FIG. 3) from Helicobacter pylori and modified metal oxide adjuvants is demonstrated by the ability to elicit protection against subsequent infection by the bacterium (reflected by the reduction of bacterium-specific 16S rDNA per gram of host tissue). Referring to FIG. 3, admixtures containing free, uncoupled modified metal oxide (3-aminopropyltriethoxysilyl α-Al₂O₃ particles) with one of the peptide surface antigens displayed protective immunity at levels considerably higher than comparable unmodified metal oxide (α-Al₂O₃ particles)-peptide covalent conjugates (as reflected by the amount of bacterium-specific 16S rDNA being proportional to the bacterial load; see FIG. 3, compare channels B vs. E and channels C vs. F). The modified metal oxide alone did not elicit bacterium-specific immunoprotection (see FIG. 3, channel A), thereby confirming that the modified metal oxide is acting as an adjuvant. Another surprising and unexpected result of this experiment is that intranasal administration of the immunological compositions is generally superior to subcutaneous administration (FIG. 3, compare channels D vs. H).

Inoculation and immunization methods and procedures via oral, i.n., i.p., i.c., and i.m. routes are well understood in the art. The applications of the adjuvant and admixtures of adjuvant with immunogen and antigenic substrates using these established procedures are within the routine practices of one skilled in the art.

The present invention contemplates pharmaceutical or immunological compositions that include modified metal oxide adjuvants in uncoupled form for administration to mammals. In a preferred embodiment, a composition for administration is a pharmaceutical or immunological composition, preferably in a single unit dosage form. Pharmaceutical or immunological compositions and single unit dosage forms can comprise a prophylacticly or therapeutically effective amount of one or more prophylactic or therapeutic agents, and a typically one or more pharmaceutically acceptable carriers or excipients or diluents.

The term “pharmaceutically acceptable” means approved by a regulatory agency of the Federal or a state government (for example, the U.S. Food and Drug Administration) or listed in the U.S. Pharmacopeia or other generally recognized pharmacopeia for use in animals, and more particularly in humans.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin. Nutraceutical compositions can, but need not, comprise one or more active or inactive ingredients that are not necessarily considered pharmaceutically acceptable to current practitioners in the art.

A pharmaceutical or immunological composition of the invention can be administered by any route according to the judgment of those of skill in the art, including but not limited to orally, intravenously, intragastrically, intraduodenally, intraperitoneally or intracerebroventricularly.

Typical pharmaceutical or immunological compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical or immunological composition or dosage form depends on a variety of factors well known in the art including, but not limited to, the way in which the dosage form will be administered to a patient and the specific active ingredients in the dosage form. The composition or single unit dosage form, if desired, can also contain minor amounts of wetting or emulsifying agents, or pH buffering agents.

The invention further encompasses administration of pharmaceutical or immunological compositions and single unit dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

The pharmaceutical or immunological compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of the immunological composition, together with a suitable amount of carrier so as to provide the form for proper administration to the patient.

The formulation should suit the mode of administration. In a preferred embodiment, the pharmaceutical or immunological compositions and single unit dosage forms are sterile and prepared in a form suitable for administration to a subject, preferably an animal subject, more preferably a mammalian subject, and most preferably a human subject. Besides humans, preferred animal subjects include horses, birds, cats, dogs, rats, hamsters, mice, guinea pigs, cows, and pigs.

A pharmaceutical or immunological composition of the invention is formulated to be compatible with its intended route of administration. Examples of routes of administration include, but are not limited to, parenteral, intravenous, intradermal, subcutaneous, intramuscular, subcutaneous, oral, buccal, sublingual, inhalation, intranasal, transdermal, topical, transmucosal, intra-tumoral, intra-synovial and rectal administration. In a specific embodiment, the composition is formulated in accordance with routine procedures as a pharmaceutical or immunological composition adapted for intravenous, subcutaneous, intramuscular, oral, intranasal or topical administration to human beings. In an embodiment, a pharmaceutical or immunological composition is formulated in accordance with routine procedures for oral administration to human beings. Typically, compositions for oral administration are solid dosage forms or solutions in sterile isotonic aqueous buffer.

Examples of dosage forms include, but are not limited to: tablets; caplets; capsules, such as soft elastic gelatin capsules or hard capsules; dropping pills; cachets; troches; lozenges; dispersions; suppositories; ointments; cataplasms (poultices); pastes; powders; dressings; creams; plasters; solutions; patches; aerosols (for example, nasal sprays or inhalers); gels; liquid dosage forms suitable for oral or mucosal administration to a patient, including suspensions (for example, aqueous or non-aqueous liquid suspensions, oil-in-water emulsions, or a water-in-oil liquid emulsions), solutions, and elixirs; liquid dosage forms suitable for parenteral or intravenous administration to a patient; and sterile solids (for example, crystalline or amorphous solids or granular forms) that can be reconstituted to provide liquid dosage forms suitable for parenteral or intravenous administration to a patient.

Pharmaceutical or immunological compositions used in the methods of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

In certain embodiments, the oral dosage forms are solid and prepared under anhydrous conditions with anhydrous ingredients, as described in detail in the sections above. However, the scope of the invention extends beyond anhydrous, solid oral dosage forms. As such, further forms are described herein.

Typical oral dosage forms are prepared by combining the modified metal oxide and immunogen or antigenic substrate in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical or immunological compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical and immunological compositions comprise from about 0.5 to about 15 weight percent of disintegrant, specifically from about 1 to about 5 weight percent of disintegrant.

Disintegrants that can be used in pharmaceutical or immunological compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical or immunological compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

The amount of the composition in the methods of the invention which will be effective in the prevention, treatment, management, or amelioration of a blood circulation disorder or one or more symptoms thereof will vary with the nature and severity of the disease or condition, and the route by which the active ingredient is administered. The frequency and dosage will also vary according to factors specific for each patient depending on the specific therapy (e.g., therapeutic or prophylactic agents) administered, the severity of the disorder, disease, or condition, the route of administration, as well as age, body, weight, response, and the past medical history of the patient. Effective doses may be extrapolated from dose-response curves derived from in vitro or animal model test systems.

In the case of liquid dosage forms, suitable concentrations of immunological compositions including the modified metal oxides are suspended or dissolved in pharmaceutically acceptable carrier media, such as water, saline, and the like. Furthermore, suitable concentrations of immunological compositions including the modified metal oxides are suspended or dissolved under physiologically and physiochemically appropriate conditions.

An effective amount of a composition described herein will provide therapeutic benefit without causing substantial toxicity. Toxicity of a composition can be determined by standard pharmaceutical procedures in cell cultures or experimental animals, for example, by determining the LD₅₀ (that is, the dose lethal to 50% of the population) or the LD₁₀₀ (that is, the dose lethal to 100% of the population).

The therapeutic index is the dose ratio between therapeutic effect and toxicity effect. Compounds that exhibit high therapeutic indices are preferred. The data obtained from these cell culture assays and animal studies can be used in formulating a dosage range that is not toxic for use in humans. The dosage of the compounds described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition and the indication to be treated. (See, for example, Fingl et al., 1996, In: The Pharmacological Basis of Therapeutics, 9.sup.th ed., Chapter 2, p. 29, Elliot M. Ross).

The descriptions of exemplary doses are merely alternative descriptions that may be used optionally at the discretion of the physician and are not intended to conflict or supersede other descriptions of doses disclosed herein. The immunological action of modified metal oxide adjuvant preparations of the invention is demonstrated by the following examples.

### EXAMPLES

### Example 1

### Preparation and Characterization of Mao

Surface Activation of the Aluminum Oxide Nanoparticles. Cleaning and surface activation of the alumina of the Al₂O₃ metal oxide nanoparticles were carried out prior to amine-modification. In a 2-L Erlenmeyer flask, 126.7 g (1.24 mol) of aluminum oxide nanoparticles (300 nm nominal diameter, calcinated at 1300° C., 99.99% pure, >95% R-form) was suspended in 1140 mL of 5% (w/v) nitric acid and heated under swirling for 90 min at 88° C. The slurry then was allowed to cool to 0° C. in an ice-water bath for 90 min before it was transferred into polyallomer centrifuge bottles, it was centrifuged at 1500×g for 10 min at 4° C., and the supernatant was aspirated. To wash the particles, they were resuspended mechanically in deionized ultrapure water at room temperature and centrifuged at 9500×g for 15 min at 4° C., and the supernatant was removed by aspiration. After a total of 10 washes in 440 mL of deionized ultrapure water each, the particle sediment was transferred into a nitric acid-cleaned glass beaker, dried at 250° C. until the weight was constant (21 h), pulverized in a nitric acid-cleaned mortar, and stored desiccated at room temperature in a nitric acid-cleaned glass bottle: yield, 112.2 g (88.6%).

Amine Modification of the Activated Aluminum Oxide Nanoparticles.

The amine modification of the alumina was accomplished in the following manner. In a 2-L round-bottom flask, 50.2 g (0.49 mol) of surface-activated, dry aluminum oxide nanoparticles was suspended in 450 mL of anhydrous toluene; 50 mL (0.21 mol) of (3-aminopropyl)-triethoxysilane was added; and the mixture was refluxed under anhydrous conditions for 23 h at 135° C. in an oil bath. Then the suspension was allowed to cool to ambient temperature over 3 h before it was transferred into polyallomer centrifuge bottles and centrifuged at 200×g for 5 min; the supernatant was aspirated. To wash the particles, they were resuspended mechanically in 450 mL of fresh toluene at room temperature and centrifuged, and the supernatant was removed by aspiration. After five washes in toluene, 450 mL each (centrifugation conditions: 200×g, 5 min, 4° C.), followed by three washes in acetone, 450 mL of each (centrifugation conditions: 5000×g, 20 min, 4° C.), the particle sediment was transferred to a nitric acid-cleaned glass beaker and dried for 17 h under vacuum at room temperature followed by 22 h at 115° C. under normal pressure. Then the sediment was pulverized in a nitric acid-cleaned mortar and the amine-modified nanoparticles were stored desiccated at room temperature in an amber bottle: yield, 48.1 g (96% referring to the weight of the underivatized surface activated particles).

The amount of free amine that was covalently linked to the aluminum oxide particles was determined with the ninhydrin method of Sarin et al. (1981). In the amine-modified MAO nanoparticles prepared above, the amine load, was 15.9 mmol of R—NH₂/g of solid.

### Example 2

### Preparation and Characterization of Other Modified Metal Oxides (Prophetic Example)

### Surface Activation of the Metal Oxide Nanoparticles

One or more metal oxides will be selected from the group consisting of titanium dioxide (TiO₂), zirconium dioxide (ZrO₂), hydroxyapatite (Ca₅(PO₄)₃(OH)), silicon dioxide (SiO₂), magnesium oxide (MgO), yttrium oxide (Y₂O₃), scandium oxide (Sc₂O₃), and lanthanum oxide (La₂O₃). Cleaning and surface activation of the metal of the metal oxide nanoparticles will be carried out prior to modification. In a 2-L Erlenmeyer flask, an amount corresponding to 1.25 mol of metal oxide nanoparticles will be suspended in 1200 mL of 5% (w/v) nitric acid and heated under swirling for 90 min at 88°-90° C. The slurry will be allowed to cool to 0° C. in an ice-water bath for 90 min prior to transfer into polyallomer centrifuge bottles. The slurry will be centrifuged at 1500×g for 10 min at 4° C., and the supernatant will be aspirated. The particles will be washed by mechanically resuspending the particles in deionized ultrapure water at room temperature. Following recollection of the particles by centrifugation at 9500×g for 15 min at 4° C., and the supernatant will be removed from the particles by aspiration. After a total of 10 washes in 500 mL of deionized ultrapure water each wash, the particle sediment will be transferred into a nitric acid-cleaned glass beaker, dried at 250° C. until the weight remains constant (20-30 h). The dried particle material will be pulverized in a nitric acid-cleaned mortar and will be stored desiccated at room temperature in a nitric acid-cleaned glass bottle.

Modification of the Activated Metal Oxide Nanoparticles.

The modification of the metal oxide will be accomplished in the following manner for surface-activated, dry metal oxide nanoparticles selected from the group consisting of titanium dioxide (TiO₂), zirconium dioxide (ZrO₂), hydroxyapatite (Ca₅(PO₄)₃(OH)), silicon dioxide (SiO₂), magnesium oxide (MgO), yttrium oxide (Y₂O₃), scandium oxide (Sc₂O₃), and lanthanum oxide (La₂O₃). In a 2-L round-bottom flask, 1.00 mol of surface-activated, dry metal oxide nanoparticles will be suspended in 450 mL of anhydrous toluene; 0.40 to 0.50 mol of 3-aminopropyl)-triethoxysilane will be added; and the mixture will be refluxed under anhydrous conditions for 23-24 h at 135° C. in an oil bath. Then the suspension will be allowed to cool to ambient temperature over 3 h before it will be transferred into polyallomer centrifuge bottles and centrifuged at 200×g for 5 min; thereafter, the supernatant will be aspirated from the particle sediment. The particles will be washed by mechanically resuspending them in 450 mL of fresh toluene at room temperature. Following centrifugation of the suspension, the supernatant will be removed by aspiration. After five washes in toluene, 450 mL each (centrifugation conditions: 200×g, 5 min, 4° C.), followed by three washes in acetone, 450 mL of each (centrifugation conditions: 5000×g, 20 min, 4° C.), the particle sediment will be transferred to a nitric acid-cleaned glass beaker and dried for at least 17 h under vacuum at room temperature followed by drying for at least 22 h at 115° C. under normal atmospheric pressure. Then the sediment will be pulverized in a nitric acid-cleaned mortar, and the amine-modified metal oxide nanoparticles will be stored desiccated at room temperature in an amber bottle. The amount of free amine that is covalently linked to the metal oxide particles will determined with the ninhydrin method of Sarin et al. (1981).

### Example 3

### Covalent Conjugating Peptides to Modified Aluminum Oxide Nanoparticles

### Method 1

Peptides will be conjugated to modified aluminum oxide nanoparticles using the experimental details provided by Frey et al. Briefly, 3-aminopropyltriethoxysilyl α-Al₂O₃ nanoparticles will be modified with homocysteinethiolactone moieties or, for example with 2-iminothiolane, and these in turn will be reacted in situ with bromoacetyl or chloroacetyl-modified peptides. For this, bromoacetic acid N-hydroxysuccinimide ester (Sigma Chemicals) will be used to modify the amino groups on the lysines with bromoacetyl moieties. In general, the synthesis of bromoacetylated peptides will proceed by reacting 1 mg/mL peptide in bicarbonate buffer, pH 8 with a 0.3× molar ratio of bromoacetic acid N-hydroxysuccinimide for 2 hr at room temperature. The bromoacetylated peptide then will be purified with HPLC and used as necessary. The amount of peptide on the Al₂O₃ nanoparticles will be monitored and quantified with the ninhydrin method. Alternative methods of conjugation of the proteins or peptides to 3-aminopropyltriethoxysilyl α-Al₂O₃ particles will be employed if necessary. One skilled in the art of organic conjugation chemistry will readily recognize effective ways to link peptides, proteins and carbohydrates to amino groups such as those on 3-aminopropyltriethoxysilyl α-Al₂O₃ particles, as well as other modified metal oxides.

### Method 2

Cysteine-containing peptides can be coupled to 3-aminopropyltriethoxy-silyl α-Al₂O₃ particles by first derivatizing 3-aminopropyltriethoxysilyl α-Al₂O₃ particles with m-maleimidobenzoyl-N-hydroxysulfosuccinimide ester, also known as sulfo-MBS from Pierce Chemical Co. The 3-aminopropyltriethoxysilyl α-Al₂O₃ particles also can be derivatized with chloroacetyl or bromoacetyl-groups. The MAO-derivatized products then can be reacted with any thiol-containing materials at neutral pH and room temperature for approximately 2 hr. The final product contains thioether-linked materials to the 3-aminopropyltriethoxysilyl α-Al₂O₃ particles.

### Example 4

### Non-Covalent Binding of LAH Peptide to MAO

The LAH peptide is derived from a highly conserved region of the influenza virus hemagglutinin surface protein and amino acid sequence is SEQ ID NO:1. [Ac-Arg-Ile-Gln-Asp-Leu-Glu-Lys-Tyr-Val-Glu-Asp-Thr-Lys-Ile-Asp-Leu-Trp-Ser-Tyr-Asn-Ala-Glu-Leu-Leu-Val-Ala-Leu-Glu-Asn-Gln-His-Thr-Ile-Asp-Leu-Thr-Asp-Ser-Glu-Met-Asn-Lys-Leu-Phe-Glu-Lys-Thr-Arg-Arg-Gln-Leu-Arg-Glu-Asn-Ala-Asp-Tyr-Lys-Asp-Asp-Asp-Asp-Lys-Cys].

One milligram LAH peptide was dissolved in 3 mL phosphate buffered saline (PBS) and the pH was checked with pH paper. The pH was found to be slightly acidic so the pH was adjusted to ca. 7.5 with the addition of 10 μL 2M NaOH. This solution then was divided into 3 equal volume aliquots. Solution 1 contained only the LAH peptide, solution 2 contained the LAH peptide and 50 mg unmodified Al₂O₃ and solution 3 contained the LAH peptide to which was added 50 mg MAO. Each solution was gently vortexed and then centrifuged at 6,000 rpm. The optical densities of the resulting supernatants then were determined at 280 nm with a Hewlett Packard Model 8451A Diode Array Spectrophotometer. The difference between the solutions would indicate the quantity of LAH bound to the insoluble Al₂O₃ particle, or to MAO (i.e., 3-aminopropyltriethoxysilyl α-Al₂O₃ nanoparticle).

TABLE 1
LAH peptide interactions with MAO

| | Metal Oxide | | |
|---|---|---|---|
| Material, | Species | O.D. (280 nm) | Amount of LAH |
| [Material] | (50 mg) | (supernatants) | Peptide Loaded |
| LAH, 0.33 mg/ml | — | 0.428 | N.D. |
| PBS | — | 0.0 | N.D. |
| LAH, 0.33 mg/ml | Al₂O₃ | 0.435 | ~0.00 |
| LAH, 0.33 mg/ml | MAO | 0.116 | 4.82 μg/mg |

Thus, 50 mg MAO binds 0.24 mg LAH peptide, or 4.8 μg/mg at pH 7.5 in PBS buffer.

Inhibition by EDTA.

In addition, ethylenediaminetetraacetic acid (EDTA) will bind strongly to MAO and inhibit the binding of anionic materials such as certain peptides, proteins or anionic carbohydrates (data not shown).

### Example 5

### Immunization of Mice with MAO−LAH Complexes

Wild type inbred C57BL/6 mice were immunized with peptide constructs identified as LAH-MAO. The peptides were prepared by a commercial source and combined with MAO by emulsion by standard means. Groups of eight mice were immunized by either s.c. or i.m. routes. Peptides were also be delivered in the absence of any adjuvant but linked covalently to Al₂O₃. One group of mice were immunized i.n. with peptide alone as a control. Finally, one group of mice remained unimmunized. Mice received a dose of 20 μg peptide on days one and 28.1.n. immunized mice received a total of 3 doses spaced 4 weeks apart. Seven days following the final booster immunization, mice were bled and antibody titers determined against the immunogen by ELISA.

Sera collected from mice immunized with the various LAH constructs were evaluated by ELISA for anti-LAH activity. 96 well microtitre plates (Nunc, PolySorp) were coated for 18 hour at 4° C. with 100 uL/well of a solution of LAH (500 ng/mL) in tris-buffered saline. This results in having 50 ng peptide per well. Wells were then washed 3 times with buffer containing 0.15M NaCl, 0.01 M sodium phosphate (PBS) and 0.1% Brij 35. The wells then are blocked with 300 uL solution containing 3% low fat dried milk and this is allowed to sit at room T for 1 h. Stock solution of the rabbit sera to be tested are composed of 20 uL serum diluted with 0.98 mL PBS Brij. Serial dilutions are made across 2 rows of the plates with each serum sample so the dilution of the sample in the first well is 1:100, second well, 1:200, 3rd well 1:400, etc. The serum samples then are incubated for 1 h at room T and then washed 3 times with PBS Brij. Then 100 uL of goat anti mouse IgG—alkaline phosphatase conjugate (Sigma) at a dilution of 1:5000 is added to each well and incubated for an hour at room T. The wells then are washed 3 times and finally treated with 100 uL/well of p-nitrophenylphosphate (PNPP) in bicarbonate/carbonate buffer pH 9. 30 minutes after the addition of the PNPP, the plates are read at 405 nm using a Thermomax Microplate Reader (Molecular Devices, Sunnyvale, Calif.). The optical density readings for the pre-bleed sera will be subtracted from the actual experimentally obtained antisera for each rabbit as a built-in correction factor to eliminate nonspecific background effects. The results will be plotted with the optical density along the y-axis and the inverse of the dilution factor along the x-axis.

### Example 6

### Non Covalent Binding of Peptides Derived from Helicobacter pylori (HP) to MAO

Peptides derived from a protein on the surface of HP were conjugated to MAO both covalently and electrostatically using the exact methods for conjugating the LAH peptide to MAO and detailed above in paragraph 85.

TABLE 2
HP-2 peptide interactions with MAO

| | Metal Oxide | | |
|---|---|---|---|
| Material, | Species | O.D. (257 nm) | Amount of HP-2 |
| [Material] | (50 mg) | (supernatants) | Peptide Loaded |
| HP-2 1.26 mg/ml | — | 0.617 | N.D. |
| PBS | — | 0.0 | N.D. |
| HP-2, .1.26 mg/ml | Al₂O₃ | 0..420 | ~0.00 |
| HP-2, 1.26 mg/ml | MAO | 0..359 | 10.5 μg/mg |

### Example 7

### Immunization of Mice with MAO−HP Peptide Complexes

The gram negative bacterium Helicobacter pylori (HP) establishes life long infection of the human gastric mucosa. Over 60% of the world's population is infected, with rates as high as 90% in developing nations. Infection results in histologic gastritis but it is also the etiologic agent of most peptic ulcer disease. HP is a risk factor for the development of gastric adenocarcinoma and it is now considered a Class I carcinogen by the WHO International Agency for Research on Cancer. The large number of infected individuals and the nature of HP-related gastric disorders creates a heavy burden on health care systems world-wide. There are no vaccines for HP.

C57BL/6 mice were immunized with MAO linked to peptide constructs derived from specific proteins found of the surface of HP. Mice were immunized intranasally and intramuscularly and they received a dose of 20 μg peptide on days one and 28. Seven days following the second immunization, mice were challenged with 107 CFU HP by gastric gavage. Mice were euthanized for analysis 4 weeks after challenge.

The final bacterial load determination was determined by quantitative PCR on frozen biopsy material. Total DNA was purified from each sample using a modification of the DNeasy DNA isolation method by Qiagen designed to purify bacterial DNA from tissue samples. Quantitative PCR was performed by comparing each sample to a standard curve utilizing serial 10-fold dilutions of purified chromosomal HP DNA. Oligonucleotide probes specific for the HP ureC gene was used.

### TERMINOLOGY AND DEFINITIONS

The terminology used herein is for the purpose of describing particular embodiments only, and is not intended to be limiting. With respect to the use of substantially, any plural and/or singular terms herein, those having skill in the art can translate from the plural as is appropriate to the context and/or application. The various singular/plural permutations may be expressly set forth herein for the sake of clarity.

As used herein, the term “modified metal oxide” refers to a metal oxide that is modified to contain a chemical moiety having positive charge at physiological pH. Thus, such modified metal oxides are cationic at physiological pH.

As used herein, the term “modified aluminum oxide” refers to an aluminum oxide that is modified to contain a chemical moiety having positive charge at physiological pH. Thus, such modified aluminum oxides are cationic at physiological pH.

The abbreviations, i.n., i.v., i.p. and s.c. refer to intranasal (or intranasally), intravenous (or intravenously), intraperitonial (or intraperitonially), and sub-cutaneous (or sub-cutaneously), respectively.

The term “pharmaceutical or immunological compositions” refers to immunological preparations, compositions or formulations that include one or more pharmaceutical components in addition to the modified metal oxide and the immunogen or antigenic substrate, as such pharmaceutical components may enhance the in vitro stability (for example, shelf-life for storage) and/or in vivo performance attributes as define by ADME properties and/or immunological responsiveness.

Terms used herein are intended as “open” terms (e.g., the term “including” should be interpreted as “including but not limited to,” the term “having” should be interpreted as “having at least,” the term “includes” should be interpreted as “includes but is not limited to,” etc.).

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “from,” “to,” “up to,” “at least,” “greater than,” “less than,” and the like, include the number recited and refer to ranges which can subsequently be broken down into subranges as discussed above.

The terms “about” and “approximately,” as such terms modify a value, refer to estimates of that value within ±25% of that value.

A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiments or examples disclosed, but that the present invention will include all embodiments falling within the scope of the appended claims.

1-88. (canceled)

89. An adjuvant agent comprising a modified metal oxide.

90. The adjuvant agent of claim 89, wherein the modified metal oxide comprises a microparticle or a nanoparticle.

91. The adjuvant agent of claim 89, wherein the modified metal oxide comprises an oxide of a metal selected from the group consisting of aluminum, iron, zinc, cadmium, cobalt, lead, chromium, titanium, zirconium, barium, calcium, magnesium, silicon, yttrium, scandium, lanthanum and mixtures thereof.

92. The adjuvant agent of claim 89, wherein the modified metal oxide comprises a linker comprising a 1-propyltriethoxysilyl or a 1-butylthriethoxysilyl moiety.

93. The adjuvant agent of claim 89, wherein the modified metal oxide comprises a chemical moiety having a positive charge at physiological pH comprising at least one member selected from the group consisting of a primary amine, a secondary amine, a tertiary amine and a quaternary amine.

94. The adjuvant agent of claim 89, wherein the adjuvant agent comprises a compound having formula (I)
X—[Y—(Z)ₘ]ₙ (I),
wherein:
X is a particle comprising a metal oxide;
Y is a linker; and
Z is a chemical moiety having a positive charge at physiological pH,
wherein:
m is an integer from 1 to 5; and
n is an integer or a fraction thereof from 0.5 to 50.

95. An immunological composition, comprising:
(a) an adjuvant agent, and
(b) an immunogen,
wherein the adjuvant agent comprises a modified metal oxide, wherein the adjuvant agent is not covalently attached to the immunogen.

96. The immunological composition of claim 95, wherein the immunogen comprises a cancer antigen, a bacteria antigen, a viral antigen or a fungal antigen.

97. The immunological composition of claim 95, wherein the modified metal oxide comprises a group of heterogeneous microparticles or nanoparticles of various sizes.

98. The immunological composition of claim 95, wherein the immunogen-metal oxide complex is selected from the group consisting of a solid, a suspension and a cream.

99. The immunological composition of claim 95, wherein the adjuvant agent comprises a compound having formula (I)
X—[Y—(Z)ₘ]ₙ (I),
wherein:
X is a particle comprising a metal oxide;
Y is a linker; and
Z is a chemical moiety having a positive charge at physiological pH,
wherein:
m is an integer from 1 to 5; and
n is an integer or a fraction thereof from 0.5 to 50.

100. A method of eliciting an immunogenic response in a host to an immunogen, comprising administering an effective amount of the immunogen, wherein said immunogen is admixed with an adjuvant agent to form a non-covalent complex between the immunogen and the adjuvant agent, and wherein the adjuvant agent comprises a modified metal oxide.

101. The method of claim 100, wherein the administering comprises intravenous injection, intraperitonial injection, sub-cutaneous injection, intramuscular injection, intraaural administration, intraocular administration, intravaginal administration, intrarectal administration or intranasal administration.

102. The method of claim 100, wherein the adjuvant agent comprises a modified metal oxide comprising a 3-aminopropyltriethoxysilyl α-Al₂O₃ nanoparticle or a 4-aminobutyltriethoxysilyl α-Al₂O₃ microparticle.

103. The method of claim 100, wherein the adjuvant agent comprises a compound having formula (I)
X—[Y—(Z)ₘ]ₙ (I),
wherein:
X is a particle comprising a metal oxide;
Y is a linker; and
Z is a chemical moiety having a positive charge at physiological pH,
wherein:
m is an integer from 1 to 5; and
n is an integer or a fraction thereof from 0.5 to 50.
